(19) United States
(12) Patent Application Publication
(10) Pub. No.: US 2009/0177082 A1
(43) Pub. Date: Jul. 9, 2009

(54) System Comprising a Gamma Probe and a Control Device

(75) Inventors: Harmut Baerwolff, Gummersbach (DE); Thomas Goebel, Berlin (DE)

Correspondence Address: SUTHERLAND ASBILL & BRENNAN LLP, 999 PEACHTREE STREET, N.E., ATLANTA, GA, 30309, US

(73) Assignee: SILICON INSTRUMENTS GMBH, Berlin (DE)

(21) Appl. No.: 12/300,903

(22) PCT Filed: May 11, 2007
(86) PCT No.: PCT/DE2007/000859

§ 371 Date: Mar. 23, 2009

(30) Foreign Application Priority Data
May 16, 2006 (DE) 10 2006 023 079.5

(51) Int. Cl. A61B 6/00

(52) U.S. Cl. 600/436

(57) ABSTRACT

The invention relates to a system comprising a medical gamma probe and a control device, which is configured for acquisition of user input for gamma probe operation, and for signal evaluation, including optional signal output in conjunction with measured signals detected by the gamma probe, wherein the medical gamma probe has a probe-side transceiver apparatus and the control device has a control device-side transceiver apparatus, between which transceiver apparatuses a wireless signal transmission connection in the form of a bidirectional radio link is formed.

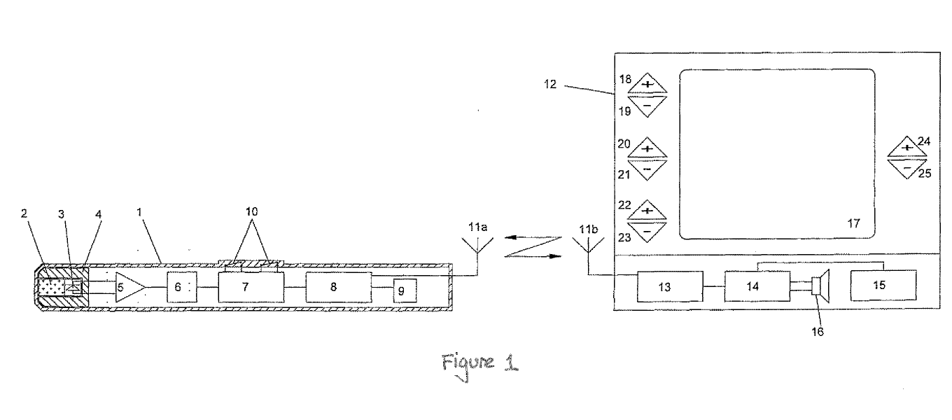

### BACKGROUND OF THE INVENTION

The invention applies to the field of medical devices for measuring radioactivity and relates in particular to a system comprising a medical gamma probe and a control device.

Radioactive marking of the first draining lymph nodes (sentinel nodes) during an operation on malignant melanoma (skin cancer) and mammary carcinoma and some cancer types in the neck and abdominal regions has become a standard method in medical practice. Radioactively labeled tumour-specific pharmaceuticals are also gaining importance due to the high spatial resolution that can be expected when directly locating cancerous tissue. The quality and duration of the search for lymph nodes or tumours depends absolutely on the technical properties and handling of the gamma probes employed for locating the radioactively marked tissue regions.

The known medical gamma probe systems used in searching for lymph nodes are cable-bound probes that are connected to an external control device for operation and signal output, as disclosed, for example, by WO 99/18853. The cable between the gamma probe and the device has a hindering effect in use and for the whole sequence of actions before, during and after the operation. This manifests itself, for example, as limited freedom of movement of the probe in the operating area, as the additional requirement for a sterile cable covering, as a trip hazard in the operating room and as the additional operating personnel needed in the non-sterile area at the control device.

Help is provided, in this regard, by a cable-free medical handheld gamma probe, as disclosed in WO 02/044755 A3. However, due to the design-related small size of the display and the operating panel, the handheld probe has disadvantages in its functional scope and in the visual and acoustic realisation of the signal output. The handheld probe is also more difficult to handle due to its size.

### SUMMARY OF THE INVENTION

It is an object of the invention to provide a system with a medical gamma probe and a control device which improves the ease of use for the operator. It is intended to avoid the disadvantages of conventional probe technology and to simplify the location of radioactively marked tissue.

This aim is achieved with a system comprising a medical gamma probe and a control device according to independent claim 1. Advantageous embodiments are the subject matter of dependent subclaims.

The invention involves the concept of a system comprising a medical gamma probe and a control device which is configured for acquisition of user input for gamma probe operation, and for signal evaluation, including optional signal output in conjunction with measurement signals detected by the gamma probe, wherein the medical gamma probe has a probe-side transceiver apparatus and the control device has a control device-side transceiver apparatus, between which transceiver apparatuses a wireless signal transmission connection in the form of a bidirectional radio link is formed.

The medical gamma probe is therefore designed to be radio-based. Signal communication between the medical gamma probe linked to the associated control device and the control device is achieved via a bidirectional radio link between the probe-side transceiver apparatus and the control device-side transceiver apparatus, which are also known as transceiver modules. By this means it has been achieved, despite the existence of an external control device, to maintain the advantages of a cable-free handheld gamma probe and, simultaneously, to provide to the user optimum user friendliness since, in operation, the medical gamma probe can be positioned without having to be connected to the control device via a signal cable. Any radio transmission technology can be used to establish the bidirectional radio link, that is, a radio link with which signals can be mutually transmitted and received, examples being ISM band (Industrial, Scientific and Medical), Bluetooth technology, wireless-LAN, ZigBee and other proprietary methods.

A convenient embodiment of the invention provides that the medical gamma probe is configured as a cable-free probe in which an energy supply module is provided for local energy supply. The embodiment as a cable-free probe signifies here that not only does the signal exchange with the control device take place via a wireless data connection, but that the probe is also not connected to any other cable, for example, an energy supply line. A battery or a rechargeable accumulator serve. for example, as the energy supply module.

In a development of the invention, it can be provided that the control device-side transceiver apparatus is connected as an external transceiver module, via an interface, to the control device. One embodiment provides, for example, the configuration of the external transceiver module as a plug-in module which is plugged into an interface on the control device and can therefore be mounted detachably.

In an advantageous embodiment of the invention, it is provided that the probe-side transceiver apparatus and the control device-side transceiver apparatus are configured to exchange signals in encrypted manner.

In a preferred development of the invention, it is provided that the probe-side transceiver apparatus and the control device-side transceiver apparatus are configured to exchange signals by means of ISM radio data transmission, optionally using a frequency band of 868/869 MHz and/or a frequency band of 902 to 928 MHz.

In advantageous embodiment of the invention, it is provided that the probe-side transceiver apparatus and the control device-side transceiver device are configured to exchange signals at a transmitting power of not more than 1 W, preferably not more than 500 mW and more preferably not more than 1 mW. This precludes harm to the health of the patient and staff by high-frequency electromagnetic radiation.

It is provided in a preferred development of the invention to assign to the probe-side transceiver and the control device-side transceiver, respectively, an unambiguous device identification number, to prevent communication by unauthorised persons.

With the aid of the proposed system with a medical gamma probe and an associated control device, a method for operating the system can be set out wherein the signals are exchanged between the medical gamma probe and the control device via the bidirectional radio link between the probe-side transceiver apparatus and the control device-side transceiver apparatus, wherein the measuring method can optionally be altered using the device-related functionality that is available.

### DESCRIPTION OF PREFERRED EMBODIMENTS OF THE INVENTION

The invention will now be described in greater detail on the basis of exemplary embodiments and making reference to the drawing.

The single figure shows a schematic representation of a system with a medical gamma probe 1 and a control device 12. For bidirectional radio transmission of signals between the cable-free medical gamma probe 1 and the control device 12, in each case, a transceiver module is provided, specifically a probe-side transceiver module 8 and a control device-side transceiver module 13. Signals of any type can be exchanged by means of the bidirectional radio link. These are, in particular, control signals, for example, signals generated by the control device 12 for controlling operation of the medical gamma probe 1, and measurement signals which indicate the measured values detected by the medical gamma probe 1 and transmitted from the medical gamma probe 1 to the control device 12 in order to process them there, for example, in the form of an acoustic or optical signal output.

In one embodiment, the control device-side transceiver module 13 can be connected via a fast data interface, preferably a USB interface, also in the form of an external module, to a control computer connected to the control device 12. The control device-side transceiver module 13 can thus be retrofitted and replaced.

The transceiver modules 8, 13 preferably operate in frequency bands that are cleared for ISM radio transmission, preferably 868/869 MHz (Europe) or 902 to 928 MHz (USA). The setting of the desired frequency band can be made, for example, using control software. In the interest of secure signal transmission, the transceiver modules 8, 13 suitably use a transmission protocol with encoded signal transmission. In order to increase transmission security, optionally CRC checksum calculation and/or internal encryption are used for the radio transmission

The medical gamma probe 1 comprises the following components shown in the drawing of the embodiment of a semiconductor detector 3 with a scintillator 2 and a collimator 4, a charge and filter amplifier 5, a threshold value or window discriminator 6, a microcontroller 7 with function keys 10 and the transceiver module 8 with a probe-side miniature antenna 11a and a power supply module 9. The power supply module 9 preferably consists of a battery or a rechargeable accumulator. By using the controller 7 with an energy-saving design and energy-optimised software and hardware management, long battery service lives are achieved.

The probe-side miniature antenna 11a of integrated design allows low transmitting and receiving power levels and thereby also low field strengths.

In the exemplary embodiment shown in the figure, the control device 12 comprises the following components: a μP-based control computer 14 with operating software, a program and data store 15 for an operating system, operating software and measurement data, a high contrast, high brightness display 17, function keys 18, . . . , 25, a loudspeaker 16 and the control device-side transceiver module 13 with a control device-side miniature antenna 11b.

All physical parameters for performing a measurement with the medical gamma probe 1 are initialised before the measurement, for example, in the context of an operation, selected on the control device 12 by a user using the function keys, or they may have been initialised in advance by means of prior settings. For user input, the control device 12 has suitable unction keys for a gate time 18, 19 or time base of the measurement value formation, nuclide preselection 20, 21 and an energy threshold or energy window 22, 23. Only the subjective parameters relating to measurement value perception, such as loudness and division factor of the acoustic signal output via the loudspeaker 16 are adjustable during measurement by means of a key press on the medical gamma probe 1. As a result, no operating personnel are needed at the control device 12 itself during the measurement.

By means of the control device 12, with further function keys 24, 25 operating modes, for example, standard measurement, background-corrected measurement or measurement with reduced statistical measuring errors, can be set before the measurement or initialised in advance by means of prior settings. The user inputs which are registered by means of different function keys are evaluated by the installed control software of the control device 12 and converted to suitable control signals.

After being switched on, the control device 12 initialises itself automatically with the parameter and operating mode pre-set values and, following automatic radio contact with the switched-on gamma probe 1, is immediately ready for operation. In standard clinical operation, therefore, no operation of the control computer 12 is necessary at all. The actual measurement value output is carried out following measurement value detection in the control device 12. The measurement values are output on the display 17 both in analogue form and digitally, as well as by acoustic signals via the loudspeaker 16.

By connecting a keyboard (not shown) to the control device 12, alphanumeric input to the software-controlled protocol creation and measurement evaluation can be carried out Measurement value storage and protocol creation can also be adjusted or initialised in advance by means of pre-set values.

The features of the invention disclosed in the above description, the claims and the figure may be significant for the realisation of the invention in its various embodiments either individually or in any combination.

1. A system comprising a medical gamma probe and a control device, which is configured for acquisition of user input for gamma probe operation, and for signal evaluation, including optional signal output in conjunction with measured signals detected by the gamma probe, wherein the medical gamma probe has a probe-side transceiver apparatus and the control device has a control device-side transceiver apparatus, between which transceiver apparatuses a wireless signal transmission connection in the form of a bidirectional radio link is formed.

2. The system according to claim 1, characterized in that the medical gamma probe is configured as a wireless probe in which an energy supply module is provided for local energy provision.

3. The system according to claim 1, characterized in that the control device-side transceiver apparatus is connected as an internal transceiver module or as an external transceiver module, via an interface, to the control device.

4. The system according to claim 1, characterized in that the probe-side transceiver apparatus and the control device-side transceiver apparatus are configured to exchange encrypted signals.

5. The system according to claim 1, characterized in that the probe-side transceiver apparatus and the control device-side transceiver apparatus are configured to exchange signals by means of ISM radio transmission, optionally using a frequency band of 868/869 MHz and/or a frequency band of 902 to 928 MHz.

6. The system according claim 1, characterized in that the probe-side transceiver apparatus and the control device-side transceiver apparatus are configured to exchange signals at a transmitting power of not more than 1 W, preferably not more than 500 mW and more preferably not more than 1 mW.
